(19) United States
(12) Patent Application Publication
(10) Pub. No.: US 2009/0016975 A1
(43) Pub. Date: Jan. 15, 2009

(54) FADE-RESISTANT COLORING COMPOSITION CONTAINING AN ACID DYE AND A CATIONIC CONDITIONING AGENT FOR A KERATIN-CONTAINING SUBSTRATE

(76) Inventors: Robert Bianchini, Hillsborough, NJ (US); Saurabh Desai, Somerset, NJ (US); Allwyn Colaco, South River, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/777,038

(22) Filed: Jul. 12, 2007

(51) Int. Cl. A61K 8/40; A61Q 1/00; A61Q 19/00; A61Q 5/10

(52) U.S. Cl. 424/63; 514/642; 8/404; 8/426

(57) ABSTRACT

The invention relates to a coloring composition for depositing colors to a keratin-containing substrate, said composition comprising: (a) one or more acid dyes; and (b) one or more cationic quaternary ammonium compounds, said quaternary ammonium compound being present in an amount which provides conditioning benefits but which is insufficient to interfere with deposition of said dye on said substrate.

### BACKGROUND OF THE INVENTION

Acid dyes provide a broad range of colors and exert little damage to hair or other materials to which they are applied. Acid dyes penetrate into the hair shaft and color the hair without simultaneous or subsequent manipulation, such as a reaction with ammonia or peroxide, thereby permitting easy and safe use of the compositions. As a result, acid dye compositions have come to be widely used as an alternative to oxidative hair dye compositions.

However, due to charge and the molecular size of acid dyes, the degree to which they penetrate into the hair fiber is quite low, making them susceptible to being readily washed out of the hair. Thus, acid dye compositions in general exhibit poor color sustainability. While solvents, such as benzyl alcohol, have been used as a penetration agent for acid dyes, solvents alone do not improve resistance of acid dyes to wash.

The isoelectric point of keratin, and more specifically of hair, is generally in the pH range of 3-4. Consequently, cationic quaternary ammonium compounds have long been used as conditioning agents in order to improve the wet and dry combability of hair. The substantivity of cationic quaternary ammonium compounds for negatively charged hair facilitates detangling during wet hair combing and a reduction in static flyaway during dry hair combing. Cationic quaternary ammonium compounds generally also impart softness and suppleness to hair. While these characteristics are desirable, cationic quaternary ammonium compounds are not generally utilized in compositions with acid dyes due to their incompatibility. Acid dyes tend to bond with cationic surfactants due to the charge to charge interaction. This substantially inhibits the dyes' penetration into hair or other surfaces. See, for example, U.S. Pat. No. 7,037,347.

### SUMMARY OF THE INVENTION

This invention relates to a coloring composition for depositing colors to a keratin-containing substrate, said composition containing: (a) one or more acid dyes; and (b) one or more cationic quaternary ammonium compounds, said quaternary ammonium compound being present in an amount which provides conditioning benefits but which is insufficient to interfere with deposition of said dye on said substrate.

This invention also relates to a method for depositing colors to a keratin-containing substrate having the following steps: providing a coloring composition containing: (a) one or more acid dyes; and (b) one or more cationic quaternary ammonium compounds, said quaternary ammonium compound being present in an amount which provides conditioning benefits but which is insufficient to interfere with deposition of said dye on said substrate; applying said coloring composition to said keratin-containing substrate for a period of time sufficient to impart said dye to the substrate; and rinsing said coloring composition from said substrate with water.

Other features and advantages of this invention will be apparent from the detailed description of the invention and from the claims.

### DETAILED DESCRIPTION OF THE INVENTION

The coloring compositions of this invention unexpectedly provide satisfactory cosmetic and physical properties, including color deposition, wet combing, dry combing, feel, manageability, body, and shine.

The compositions and methods of this invention utilize a concentration range of cationic quaternary ammonium compounds that are surprisingly and unexpectedly compatible with acid dyes and provide superior coloring and conditioning benefits. It is both surprising and unexpected for the coloring compositions of this invention to be consumer-appealing, to maintain product stability over long storage times, and to impart excellent hair coloring and conditioning properties to treated hair. Furthermore, the coloring compositions of this invention are sufficiently fade resistant to wash.

The compositions of this invention can be applied to the hair at ambient temperature and is allowed to contact the hair for a relatively short period of time to provide hair coloring and conditioning. Consequently, the method and composition of this invention color and condition the hair to provide a more manageable and esthetically-pleasing hair treatment experience.

It is believed that one skilled in the art can, based upon the description herein, utilize the compositions and methods of this invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference. Unless otherwise indicated, a percentage refers to a percentage by weight (i.e., % (W/W)).

### Keratin-Containing Substrate

For the purpose of this invention, the term “keratin-containing substrate” includes hair, skin, nails, teeth, tissues, wool, fur, and any other materials that contain keratin proteins. Keratin-containing substrate of this invention is preferably human hair, skin, or nail.

Human hair used in the examples below available commercially, for example from International Hair Importers and Products (Bellerose, N.Y.), in different colors, such as brown, black, red, and blond, and in various types, such as African-American, Caucasian, and Asian. Additionally, the hair samples may be chemically treated.

### Conditioning Benefits:

Conditioning benefits include, but are not limited to, combability and shine. Combability is defined as ease of dragging a comb through hair. This force is measured using a Diastron combing apparatus available from Diastron Corporation, Hampshire, UK. Preferably, said force is less than about 0.2 joules. Shine is measured with a shine box, which is a black box that has hair mounted on a cylinder of known curvature where reflected light is captured and measured. Multiple measurements are then taken on the light that is reflective from the hair and a shine value is calculated. (See Robbins C. R., Light scattering and shine measurements of human hair: a sensitive probe of the hair surface, J. Soc. Cosmet. Chem., 1993, 44:221). Sufficient conditioning benefits provide hair with a reduction in frictional force. A 50% reduction of combing force is typical for bleached or damaged hair. Hair that is conditioned is considered to be in a proper and healthy state, easier to manage and more appealing to the consumers.

### Color Deposition

Color deposition can be measured by placing human hair swatches in a Hunter Labs color spectrophotometer. Color deposition is characterized by the parameter “Delta E”, which is the measurement of total color change using the L,a,b scale, wherein L* is lightness units, a* is red-green units, and b* is yellow-blue units. When a red dye is applied to hair, the a* value represents the amount of redness on the hair, and the Delta a* value is directly correlated to the amount of red dye that is deposited on the hair. Sufficient color deposition is perceived when a Delta E of at least 2 units is achieved. See Richard S. Hunter, Richard W. Harold, “The Measurement of Appearance”, Second Edition, John Wiley & Sons, (1987).

### Acid Dyes:

Acid dyes (also known as “anionic dyes”) are natural or synthetic organic compounds whose molecules contain two groups of atoms, one acidic group, and one color-producing group. Acid dyes that are useful in the compositions of this invention are water-soluble acid dyes commonly used in classical cosmetic compositions, as set forth, for example, in Green et al. CFTA International Color Handbook, 2nd ed., Micelle Press, England (1992) and Cosmetic Handbook, US Food and Drug Administration, FDA/IAS Booklet (1992). The acid dyes useful in the compositions of this invention are available commercially from various sources such as Sensient Technologies, Sun Chemicals, and Lowenstein Chemicals. Suitable acid dyes include, but are not limited to, Acid Black 1, Acid Blue 1, Food Blue 5, Acid Blue 9, Acid Blue 74, Acid Red 18, Acid Red 27, Acid Red 87, Acid Red 92, Acid Orange 7, Acid Violet 43, Acid Yellow 1, Acid Yellow 23, Acid Yellow 3, Food Yellow 8, D & C Brown 1, D & C Green 5, D & C Orange 4, D & C Orange No. 10, D & C Orange 11, D & C Red 21, D & C Red 27, D & C Red 33, D & C Yellow 7, D & C Yellow 8, FD & C Red 4, FD & C Yellow 6, and the mixtures thereof. The preferred acid dyes of this invention are acid black 1, Red 33, acid yellow 23, and the mixtures thereof.

The acid dyes useful in the compositions of this invention may be used either singly or in any combination thereof in order to achieve the desired color. Preferably, they are present in an amount just sufficient to impart color to the substrate to which they are applied. Preferably, the acid dye useful in the compositions of this invention is present in the composition at a concentration range from about 0.01% to about 5% by weight. More preferably, the acid dye is present in the composition at a concentration range from about 0.5% to about 3% by weight. Most preferably, the acid dye is present in the composition at a concentration range from about 1% to about 2% by weight.

### Cationic Quaternary Ammonium Compounds

The cationic quaternary ammonium compounds useful in the compositions and methods of this invention are preferably composed of a non-polymeric molecule having at least one C₈-C₃₆ carbon chain and one positively charged quaternary nitrogen atom per molecule. The cationic quaternary ammonium compounds useful in the compositions of this invention may have one or more C₈-C₃₆ alkyl or alkenyl carbon chain groups. The quaternary ammonium compounds useful in the compositions and methods of this invention preferably have more than one C₈-C₃₆ alkyl or alkenyl carbon chain groups. Particularly preferred are quaternary ammonium compounds having three C₈-C₃₆ alkyl or alkenyl carbon chain groups.

The quaternary ammonium compounds of this invention can have branched carbon chains, unbranched carbon chains or mixtures of branched and unbranched carbon chains. The remaining substitutes of the quaternary nitrogen of the quaternary ammonium compound can be hydrogen, benzyl, short chain alkyl such as methyl or ethyl group, hydroxyalkyl groups such as hydroxymethyl or hydroxyethyl group, or the mixtures thereof.

The cationic quaternary ammonium compounds useful in the compositions of this invention can be substantially or partially water-soluble. The cationic quaternary ammonium compounds useful in the compositions of this invention can also be substantially or partially water-dispersible. The cationic quaternary ammonium compounds useful in the compositions of this invention can be a liquid or solid.

The cationic quaternary ammonium compounds may have any desired charge-balancing anions, but preferably are associated with chloride, iodide, bromide, methosulfate, hydrogensulfate, lactate or citrate, particularly preferably chloride and methosulfate counter ions.

The cationic quaternary ammonium compounds useful in the compositions and methods of this invention may be prepared in a known manner by alkylation of a tertiary amine in the presence of at least one unbranched or branched mono alkyl group having from one to four carbon atoms. Preferably, the quaternary ammonium compound useful in the compositions and methods of this invention are in the form of a pellet or, more preferably, a powder.

The compositions of this invention may contain one or more cationic quaternary ammonium compounds. The quaternary ammonium compounds are present in an amount sufficient to provide conditioning benefits, yet insufficient to interfere with the ability of the acid dyes present in the compositions to be deposited on and penetrate into the hair. The cationic quaternary ammonium compounds of this invention may contain one C₈-C₃₆ alkyl or alkenyl group. Examples of such cationic quaternary ammonium compounds include, but are not limited to, behentrimonium methosulfate, behentrimonium chloride, cetyl trimethyl ammonium chloride, soytrimonium chloride, and the mixtures thereof. The quaternary ammonium compound containing one C₈-C₃₆ alkyl or alkenyl group is preferably present in the concentration range from about 0.1% to about 3% by weight, and more preferably from 0.5% to about 2% by weight.

Preferably, the cationic quaternary ammonium compounds of this invention contain two C₈-C₃₆ alkyl or alkenyl groups. The quaternary ammonium compounds include, but are not limited to, dilauryldimonium chloride, distearyldimonium chloride, dicetyldimonium chloride, dibehenyldimonium chloride, and the mixtures thereof. The quaternary ammonium compound containing two C₈-C₃₆ alkyl or alkenyl groups is preferably present in the concentration from about 0.1% to about 1% by weight, more preferably from 0.3% to about 0.7% by weight, and most preferably about 0.5% by weight.

Most preferably, the cationic quaternary ammonium compounds of this invention have three C₈-C₃₆ alkyl or alkenyl groups. The quaternary ammonium compound includes, but is not limited to trilaurylammonium chloride, tricetylammonium chloride, tristearyl ammonium chloride, and the mixtures thereof. The quaternary ammonium compound containing two C₈-C₃₆ alkyl or alkenyl group is preferably present in the concentration from about 0.1% to about 1% by weight, more preferably from 0.3% to about 0.7% by weight, and most preferably about 0.5% by weight.

### Color Enhancing Solvent:

The compositions of this invention preferably further contain one or more organic solvents that function to enhance the penetration of acid dyes into the substrate that is to be dyed. Such color enhancing solvents include, but are not limited to, non-aromatic solvents, aromatic solvents and mixtures thereof. Examples of color enhancing aromatic solvents include, but are not limited to, benzyl alcohol, cinnamyl alcohol, phenethyl alcohol, p-anisyl alcohol, p-methylbenzyl alcohol, 2-benzyloxyethanol, phenoxy ethanol, and the mixtures thereof. More preferably, the color enhancing aromatic solvents useful in the compositions of this invention are benzyl alcohol, phenoxy ethanol and the mixtures thereof.

The color enhancing non-aromatic solvents of this invention include, but are not limited to, ethanol, isopropanol, acetone, methoxyethanol, propylene glycol, propan-1,2-diol, butylene glycol, dimethoxy methane, methoxy-isopropanol, diethylene glycol, butan-1,4-diol, diethylene glycol monoethylenether, pentan-1,2-diol, diethylene glycol monoethylether, dipropylene glycol, dipropylene glycol monomethylether, and the mixtures thereof.

The color enhancing solvents useful in the compositions of this invention may be used either singly or in any combination. They should preferably be present in the compositions of this invention in an amount sufficient to improve penetration of the acid dyes into the substrate that is being dyed. For example, preferably, the color enhancing solvent should be present in the composition at a concentration range from about 0.01% to about 10% by weight. More preferably, the color enhancing solvent should be present in the composition at a concentration range from about 0.5% to about 5% by weight.

### Additional Conditioning Agents

Additional conditioning agents may be provided in the compositions of this invention to impart improved characteristics to the substrates being dyed. These can include conditioning agents that improve the appearance, texture, luster, and sheen of the substrates as well as increasing the substrates' body or suppleness. While the conditioning agents mentioned below relate to hair conditioning, such agents may also be utilized when dyeing fabric, nails, or other substrates to improve texture and appearance of the substrates.

The additional conditioning agents can also be included in the coloring compositions of this invention to facilitate the composition formulation and to enhance consumer application. The preferred additional conditioning agents of this invention are those that are nonionic or anionic. Suitable examples of additional hair conditioning agents include, but are not limited to, fatty alcohols; fatty amines; waxes; nonionic esters; polymers, such as polyvinylpyrolidone, polyvinyl alcohol, and polyethylene glycol; silicones; siloxanes; and polymer emulsions. Examples of preferred additional conditioning agents of this invention include, but are not limited to, non-ionic silicone conditioning agents, anionic silicone conditioning agents, non-ionic surfactant conditioning agents, anionic surfactant conditioning agents or the mixtures thereof.

Examples of non-ionic silicone conditioning agents include, but are not limited to, silicone gums, dimethicone co-polyol, silicone elastomers, cyclomethicone, phenyl methicone, dimethyl polysiloxane, methylphenyl polysiloxane, amino-modified silicones, alkyl-modified silicones, and the mixtures thereof.

Examples of anionic silicone conditioning agents include, but are not limited to, anionic silicone resin, PEG dimethicone succinate, sodium salts of esters of dimethicone copolyol and sulphosuccinic acid, silicone phosphate esters, and the mixtures thereof.

Non-ionic surfactant conditioning agents include, but not are limited to, stearylamidopropyl dimethylamine, isostearamidopropyl dimethylamine, oleamidopropyl dimethylamine, behenaamidopropyl dimethylamine, brassicamidopropyl dimethylamine, didecylmethylamine oxide, stearyldimethylamine oxide, and the mixtures thereof.

Anionic surfactant conditioning agents include, but not are limited to, fatty acids, fatty acid sulfates, fatty acid sulfonates, and the mixtures thereof.

### Additional Quaternary Ammonium Compound

Other optional quaternary ammonium compounds can be included in the coloring compositions of this invention to enhance the color deposition and conditioning benefits of the compositions. For example, an oil-soluble, water dispersible quaternary ammonium compound, either alone or in combination with a water-soluble quaternary ammonium compound, also can be used in the composition of this invention.

### Optional Thickeners

An optional thickener may also be included in the compositions of this invention to improve composition esthetics and facilitate application of the composition to the substrates being dyed. Nonionic thickeners in an amount of 0.01% to up to about 5% by weight are preferred. Exemplary thickeners include, but are not limited to, celluloses or celluloses derivatives, guar gum or guar gum derivatives, Xanthan gum, polysaccharides, acrylamides copolymer, acrylates/behenth-25, methacrylate copolymer, acrylates C10 30 alkyl acrylate crosspolymer, acrylates ceteth-20 itaconate copolymer, acrylates/steareth-50 acrylate copolymer, acrylates/stearyl methacrylate copolymer, acrylates/vinyl isodecanoate crosspolymer, and the mixtures thereof.

### Other Cosmetic Components and Additives

In addition to the above-described ingredients, other common cosmetic components and additives known or otherwise effective for use in hair care or personal care products may be incorporated in the compositions of this invention, as long as the basic properties of the compositions, and the ability to color substrates, are not adversely affected. Such optional ingredients include, but are not limited to, anti-dandruff agents, hair growth agents, anti-inflammatory agents, anti-microbial agents, anionic and nonionic surfactants, suspending Agent humectants, emollients, moisturizers, fragrances, additional dyes and colorants, foam stabilizers, anti-static agents, preservatives, rheology modifiers, water softening agents, chelants, hydrotropes, polyalkylene glycols, acids, bases, buffers, beads, pearlescent aids, fatty alcohols, proteins, skin active agents, sunscreens, vitamins, and pediculocides, and the like. Optional components may be present in weight percentages of less than about 1% each, and from about 0.01% to about 10% by weight of the composition in total.

### Cosmetically Acceptable Carriers:

The compositions of this invention preferably contain one or more cosmetically-acceptable carriers. Preferably, such carriers include water. Organic solvents may also be included in order to facilitate manufacturing of the compositions or to provide esthetic properties, such as viscosity control. Suitable solvents include the lower alcohols, such as ethyl alcohol and isopropyl alcohol; glycol ethers, such as 2-butoxyethanol, ethylene glycol monoethyl ether, propylene glycol and diethylene glycol monoethyl ether or monomethyl ether; and the mixtures thereof. Non-aqueous solvents may be present in the coloring compositions of this invention in an amount of about 0.01% to about 50%, and in particular about 1% to about 20%, by weight of the total weight of the carrier in the compositions.

The compositions of this invention should be stable to phase or ingredient separation at a temperature of about 25° C. for a long period of time, or at least for about 26 weeks at a temperature of between 4° C. and 40° C. Thus, the compositions of this invention have demonstrated sufficient stability to phase and ingredient separation at temperatures normally found in commercial product storage and shipping to remain unaffected for a period of at least six months.

This invention also relates to methods of using the compositions of this invention to dye keratin-containing substrates, including hair. Although the following recites hair as the substrate to be dyed, the method described herein may be applied to other keratin-containing substrates that are amenable to dyeing with acid dyes. Treatment of hair with the compositions of this invention is generally carried out by: (1) applying to dry or wet hair an sufficient amount of the coloring composition of the invention; (2) distributing the composition of this invention more or less evenly throughout the hair such that it contacts all the hair or other substrates which is intended to be colored. This permits the acid dyes of the compositions of this invention to penetrate into the hair or other keratin-containing substrate. This step may be accomplished by rubbing the composition throughout the hair manually or using a hair appliance such as a comb or a brush for up to about two to about twenty minutes; and (3) rinsing said hair or other substrates so as to remove excess material that has not penetrated into the hair with water. Treatment of hair with the compositions of the invention may also be carried out by applying leave-on types of compositions of this invention, such as hair spray, cream, foam, or solution, directly to hair without rinsing the hair.

The coloring compositions of this invention may be used to deposit colors on previously colored hair. The previous coloring compositions generally do affect the color deposition on the hair. The compositions of this invention employ water soluble agents that can be easily rinsed from the hair, while enabling the acid dyes of the compositions of this invention to be effectively deposited on the hair.

The steps for preparing the compositions of this invention may include, but are not limited to the following steps: (1) Heat the water to from about 50° C. to about 70° C. Optionally add a thickening agent to the water and mix for about 2 to 30 minutes or until the mixture is uniform. Heat the mixture again to from about 70° C. to 90° C.; (2) Add a cationic quaternary ammonium compound to the mixture and mix it for about 20 minutes or until the mixture is uniform; (3) Cool the mixture to from about 40° C. to about 30° C. (4) Add dye, solvent and additives to the mixture and mix it again until the mixture is uniform.

Other optional ingredients can be added during or after the above steps as long as they do not decrease the coloring and conditioning performance of the composition of this invention.

The pH of the compositions of this invention is within the range of from about 3 to about 11, and preferably within the range from about 5 to about 8. Additionally, it should be noted that the pH of the compositions of this invention may be adjusted in accordance with a method known to those of skill in the art. Maintaining the pH in the stated range facilitates the penetration of acid dye into the hair. The acid used in the adjustment of the pH is preferably a weak acid because the hair itself has an ion exchange capability. Specific examples thereof include organic acids such as citric acid, glycolic acid, succinic acid, tartaric acid, lactic acid, fumaric acid, malic acid, levulinic acid, butyric acid, valeric acid, oxalic acid, maleic acid and mandelic acid, and inorganic acids such as phosphoric acid. It is preferred that each of these weak acids be used in combination with its potassium salt, sodium salt, ammonium salt, triethanolamine salt or the like to impart a buffer capacity to the system.

The buffer capacity is preferably not lower than 0.007 gram equivalents/liter, but lower than 0.2 gram equivalents/liter, preferably not lower than 0.01 gram equivalents/liter, but lower than 0.2 gram equivalents/liter, more preferably not lower than 0.015 gram equivalents/liter, but lower than 0.2 gram equivalents/liter as the buffer capacity of a 10% aqueous solution of the hair dye composition. The term “buffer capacity” used in the context of the compositions of this invention means a value determined by using, as a measurement, the concentration of a base required to raise the pH of a 10% aqueous solution by 1 from the initial value.

The amount of compound incorporated for imparting such a buffer capacity is not particularly specified and varies according to the kind of the compound for imparting the buffer capacity. For example, when sodium citrate is used as the compound for imparting the buffer capacity, the compound is incorporated at a concentration of at least about 1%.

The compositions and methods of this invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### EXAMPLE 1

### Formulation

Tables 1 and 2 illustrate two of the coloring compositions of this invention. The steps for preparing the compositions set forth in Table 1 were as follows:

Step 1. The water was heated in a vessel to 60° C. Hydroxyethylcellulose was added to the water. The combination was mixed until it was uniform. The combination was again heated to 80° C.

Step 2. Cetyl Trimethyl Ammonium Chloride was added to the mixture. The combination was mixed until it was uniform.

Step 3. The mixture was cooled to 60° C. Dimethicone (50 cs) was added to the mixture and the combination stirred until uniform. The mixture was again cooled to about room temperature.

Step 4. D&C Acid Red #33 and fragrance were added to the composition at room temperature and mixed until uniform.

TABLE 1

| Ingredients | Wt. % | Sources of materials |
|---|---|---|
| Water | 97 | |
| Hydroxyethylcellulose | 1.5 | The Dow Chemical |
| | | Company |
| | | Dow Center |
| | | Midland, MI 48674 |
| D&C Acid Red #33 | 1.0 | Sensient |
| | | Milwaukee, WI 53202 |
| Cetyl Trimethyl | 0.5 | Croda |
| Ammonium Chloride | | Edison, NJ 08837 |
| Dimethicone | 0.5 | The Dow Corning Company |
| | | Midland, MI 48674 |
| Fragrance | 0.2 | Firmenich |
| | | Princeton, NJ 08543 |

TABLE 2

| Ingredients | Wt. % | Sources of materials |
|---|---|---|
| Water | 96 | |
| Hydroxyethylcellulose | 1.5 | The Dow Chemical |
| | | Company |
| | | Dow Center |
| | | Midland, MI 48674 |
| D&C Acid Red #33 | 1.0 | Sensient |
| | | Milwaukee, WI 53202 |
| Behenyl Trimethyl | 1.5 | Croda |
| Ammonium Chloride | | Edison, NJ 08837 |
| Dimethicone | 0.5 | The Dow Corning Company |
| | | Midland, MI 48674 |
| Fragrance | 0.2 | Firmenich |
| | | Princeton, NJ 08543 |

TABLE 3

| | Sources of | Composition 1 | Composition 2 | Composition 3 |
|---|---|---|---|---|
| Ingredients | materials | (Wt. %) | (Wt. %) | (Wt. %) |
| Water | | 91.0 | 90.5 | 90.0 |
| Hydroxyethylcellulose | The Dow | 1.5 | 1.5 | 1.5 |
| | Chemical | | | |
| | Company | | | |
| | Dow Center | | | |
| | Midland, MI | | | |
| | 48674 | | | |
| D&C Acid Red | Sensient | 1.0 | 1.0 | 1.0 |
| #33 | Milwaukee, WI | | | |
| | 53202 | | | |
| Cetyl | Croda | 0.5 | 1.0 | 1.5 |
| Trimethyl | Edison, NJ | | | |
| Ammonium | 08837 | | | |
| Chloride | | | | |

### EXAMPLE 2

### Color Deposition of Acid Dye with Variation in Concentration and the Alkyl Chain Length of the Cationic Quaternary Ammonium Compounds

Tables 4-7 illustrate the color deposition of Red #33 with various levels of the cationic quaternary ammonium compounds, Behentrimonium Methosulfate, Behentrimonium Chloride, Cetyl Trimethyl Ammonium Chloride, and Lauryl Trimethyl Ammonium Chloride, respectively.

The acid dye used in this example was obtained from Sensient Technologies (CAS # 3567-66-6, empirical formula C16H13N3O7S2.2Na, Milwaukee, Wis. 53202). The cationic quaternary ammonium compounds used in this example were obtained from Croda (Edison, N.J. 08837). The human hair sample used in this example was Caucasian blond obtained from International Hair Importers and Products (Bellerose, N.Y.).

The a* values shown in Tables 4-7 represent the amount of total redness on each hair sample, respectively, and they were independent to each other. Furthermore, the a* values were directly correlated to the amount of red dye that was deposited on the hair samples.

TABLE 4
Color deposition of acid dye with
Behentrimonium Methosulfate

| | Sam- | Sam- | | | |
|---|---|---|---|---|---|
| Ingredients | ple 1 | ple 2 | Sample 3 | Sample 4 | Sample 5 |
| Red #33 | 1.0 | 1.0 | 1.0 | 1.0 | 1.0 |
| HEC | 1.5 | 1.5 | 1.5 | 1.5 | 1.5 |
| Behentrimonium | 0.25 | 0.5 | 1.0 | 1.5 | 2.0 |
| Methosulfate | | | | | |
| water | qs | qs | qs | qs | qs |
| a* value after dye | 28.75 | 27.34 | 19.58 | 18.69 | 15.25 |
| deposition | | | | | |

TABLE 5
Color deposition of acid dye with
Behentrimonium Chloride

| | Sam- | Sam- | | | |
|---|---|---|---|---|---|
| Ingredients | ple 1 | ple 2 | Sample 3 | Sample 4 | Sample 5 |
| Red #33 | 1.0 | 1.0 | 1.0 | 1.0 | 1.0 |
| HEC | 1.5 | 1.5 | 1.5 | 1.5 | 1.5 |
| Behentrimonium | 0.25 | 0.5 | 1.0 | 1.5 | 2.0 |
| Chloride | | | | | |
| water | qs | Qs | qs | qs | qs |
| a* value after | 26.42 | 28.48 | 24.12 | 10.49 | 9.54 |
| Dye deposition | | | | | |

TABLE 6
Color deposition of acid dye with
Cetyl Trimethyl Ammonium Chloride

| Ingredients | Sample 1 | Sample 2 | Sample 3 | Sample 4 | Sample 5 |
|---|---|---|---|---|---|
| Red #33 | 1.0 | 1.0 | 1.0 | 1.0 | 1.0 |
| HEC | 1.5 | 1.5 | 1.5 | 1.5 | 1.5 |
| Cetyl | 0.25 | 0.5 | 1.0 | 1.5 | 2.0 |
| Trimethyl | | | | | |
| Ammonium | | | | | |
| Chloride | | | | | |
| water | Qs | qs | qs | qs | qs |
| a* value | 28.5 | 25.85 | 15.55 | 16.66 | 16.19 |
| after dye | | | | | |
| deposition | | | | | |

TABLE 7
Color deposition of acid dye with
Lauryl Trimethyl Ammonium Chloride

| Ingredients | Sample 1 | Sample 2 | Sample 3 | Sample 4 | Sample 5 |
|---|---|---|---|---|---|
| Red #33 | 1.0 | 1.0 | 1.0 | 1.0 | 1.0 |
| HEC | 1.5 | 1.5 | 1.5 | 1.5 | 1.5 |
| Lauryl | 0.25 | 0.5 | 1.0 | 1.5 | 2.0 |
| Trimethyl | | | | | |
| Ammonium | | | | | |
| Chloride | | | | | |
| water | qs | qs | qs | qs | qs |
| a* value | 28.68 | 28.71 | 18.38 | 20.25 | 19.39 |
| after dye | | | | | |
| deposition | | | | | |

*Q.S. = remaining balance adding to 100%,
a* value = total redness on each hair sample, respectively, and they were independent to each other.
higher a* value = increase in red tone,
lower a* value = decrease in red tone.

In Table 4, the a* value after dyeing using the coloring composition containing the cationic quaternary ammonium compound, Behentrimonium Methosulfate, remained above or near 20 until the concentration of Behentrimonium Methosulfate reached above 1.5% by weight. The visual inspections of the hair samples indicated that when the concentration of Behentrimonium Methosulfate reached 2%, the color uptake of the hair samples decreased significantly. At 2% of the Behentrimonium Methosulfate concentration, the hair sample exhibited little color uptake.

In Table 5, the a* value after dyeing using the coloring composition containing the cationic quaternary ammonium compound, Behentrimonium Chloride, remained above or near 25 until the concentration of Behentrimonium Chloride reached above 1.5% by weight. The visual inspections of the hair samples indicated that when the concentration of Behentrimonium Chloride reached 1.5%, the color uptake of the hair samples decreased significantly. At 1.5% and 2% of the Behentrimonium Chloride concentrations, the hair samples exhibited little color uptake.

In Table 6, the a* value after dyeing using the coloring composition containing the cationic quaternary ammonium compound, Cetyl Trimethyl Ammonium Chloride, remained above 25 until the concentration of Behentrimonium Chloride reached 1% by weight. The visual inspections of the hair samples indicated that when the concentration of Cetyl Trimethyl Ammonium Chloride reached 1%, the color uptake of the hair samples decreased significantly.

In Table 7, the a* value after dyeing using the coloring composition containing the cationic quaternary ammonium compound, Lauryl Trimethyl Ammonium Chloride, remained above or near 20. However, the a* values decreased significantly when the concentration of Lauryl Trimethyl Ammonium Chloride reached above 1% by weight. The visual inspections of the hair samples indicated that when the concentration of Lauryl Trimethyl Ammonium Chloride reached 1%, the color uptake of the hair samples decreased significantly.

The results in Tables 4-7 show that the color deposition of Red #33 was sufficient and remained relatively stable within certain concentration ranges of the cationic quaternary ammonium compounds. Above such an optimal range, the color deposition declined rapidly with the increased concentration of the cationic quaternary ammonium compounds.

Tables 4-7 also show that varying the alkyl chain length from 22 carbon atoms (Behentrimonium) to 16 carbon atoms (Cetyl Trimethyl) and to 12 carbon atoms (Lauryl Trimethyl) of the cationic quaternary ammonium compounds did not significantly affect the color deposition. In addition, the choice of counter ions of the cationic quaternary ammonium compounds also did not have a significant effect on the color deposition

### EXAMPLE 3

### The Conditioning Effect of the Coloring Compositions with Variation in Concentration of the Cationic Quaternary Ammonium Compound

Table 8 illustrates the wet combing force (frictional force) of the hair treated with the coloring compositions with variation in concentration of the cationic quaternary ammonium compound, Behentrimonium Chloride. The results in Table 8 show that at 0.5% of the cationic quaternary ammonium compound concentration, the frictional force to run a comb though the hair samples was reduced by 50% or more compared to the sample without the cationic quaternary ammonium compound. Additionally, the total combing force was further reduced when 0.25% silicone fluid was added. Therefore, it can be concluded that the coloring compositions of this invention provided sufficient conditioning benefits.

TABLE 8

| | Frictional |
|---|---|
| Composition | force (J) |
| hydroxyethylcellulose & water | 0.31 |
| 0.5% Behentrimonium Chloride + hydroxyethylcellulose & | 0.18 |
| water | |
| 1.0% Behentrimonium Chloride + hydroxyethylcellulose & | 0.13 |
| water | |
| 1.5% Behentrimonium Chloride | 0.09 |
| 0.5% Behentrimonium Chloride + 0.25% | 0.08 |
| Dimethicone + hydroxyethylcellulose & water | |

What is claimed is:

1. A coloring composition for depositing colors to a keratin-containing substrate, said composition comprising:
a) one or more acid dyes;
b) one or more cationic quaternary ammonium compounds, said quaternary ammonium compound being present in an amount which provides conditioning benefits but which is insufficient to interfere with deposition of said dye on said substrate.

2. The coloring composition of claim 1, wherein said quaternary ammonium compound has one C₈-C₃₆ alkyl or alkenyl group.

3. The coloring composition of claim 2, wherein said quaternary ammonium compound is selected from the group consisting of behentrimonium methosulfate, behentrimonium chloride, cetyl trimethyl ammonium chloride, soytrimonium chloride, and the mixtures thereof.

4. The coloring composition of claim 2, wherein said quaternary ammonium compound has a concentration range from about 0.1% to about 3% by weight.

5. The coloring composition of claim 4, wherein said quaternary ammonium compound has a concentration range from about 0.5% to about 2% by weight.

6. The coloring composition of claim 5, wherein said quaternary ammonium compound has a concentration range from about 1% to about 2% by weight.

7. The coloring composition of claim 1, wherein said quaternary ammonium compound has two or three C₈-C₃₆ alkyl or alkenyl groups, or the mixtures thereof.

8. The coloring composition of claim 7, wherein said quaternary ammonium compound is selected from the group consisting of Dilauryldimonium Chloride, Distearyldimonium Chloride, Dicetyldimonium Chloride, Dibehenyldimonium Chloride, trilaurylammonium chloride, tricetylammonium chloride, tristearylammonium chloride, and the mixtures thereof.

9. The coloring composition of claim 7, wherein said quaternary ammonium compound has a concentration range from about 0.1% to about 1% by weight.

10. The coloring composition of claim 9, wherein said quaternary ammonium compound has a concentration range from about 0.3% to about 0.7% by weight.

11. The coloring composition of claim 10, wherein said quaternary ammonium compound has a concentration range from about 0.5% by weight.

12. The coloring composition of claim 1, wherein said acid dye is selected from the group consisting of Acid Black 1, Acid Blue 1, Food Blue 5, Acid Blue 9, Acid Blue 74, Acid Red 18, Acid Red 27, Acid Red 87, Acid Red 92, Acid Orange 7, Acid Violet 43, Acid Yellow 1, Acid Yellow 23, Acid Yellow 3, Food Yellow 8, D & C Brown 1, D & C Green 5, D & C Orange 4, D & C Orange No. 10, D & C Orange 11, D & C Red 21, D & C Red 27, D & C Red 33, D & C Yellow 7, D & C Yellow 8, FD & C Red 4, FD & C Yellow 6, and the mixtures thereof.

13. The coloring composition of claim 12, wherein said acid dye is selected from the group consisting of acid black 1, Red 33, acid yellow 23, and the mixtures thereof.

14. The coloring composition of claim 1, further comprising one or more color enhancing solvents selected from the group consisting of non-aromatic solvents, aromatic solvents and the mixtures thereof.

15. The coloring composition of claim 14, wherein said color enhancing aromatic solvent is selected from the group consisting of benzyl alcohol, cinnamyl alcohol, phenethyl alcohol, p-anisyl alcohol, p-methylbenzyl alcohol, 2-benzyloxyethanol, Phenoxy ethanol, and the mixtures thereof.

16. The coloring composition of claim 14, wherein said color enhancing aromatic solvent is selected from the group consisting of benzyl alcohol, phenoxy ethanol and the mixtures thereof.

17. The coloring composition of claim 1, further comprising one or more thickening agents.

18. The coloring composition of claim 17, wherein said thickening agent is selected from the group consisting of celluloses or celluloses derivatives, guar gum or guar gum derivatives, polysaccharides, acrylamides copolymer, acrylates/behenth-25, methacrylate copolymer, acrylates C10 30 alkyl acrylate crosspolymer, acrylates ceteth-20 itaconate copolymer, acrylates/steareth-50 acrylate copolymer, acrylates/stearyl methacrylate copolymer, acrylates/vinyl isodecanoate crosspolymer, and the mixtures thereof.

19. The coloring composition of claim 1, further comprising one or more additional conditioning agents selected from the group consisting of silicones, siloxanes, fatty acids, fatty alcohols, fatty esters, fatty ethers and the mixtures thereof.

20. A method for depositing colors to a keratin-containing substrate comprising: providing a coloring composition; applying said coloring composition to said keratin-containing substrate for 1 to 20 minutes; and rinsing said coloring composition from said substrate with water;
wherein said coloring composition comprising:
a) one or more acid dyes;
b) one or more cationic quaternary ammonium compounds, said quaternary ammonium compound being present in an amount which provides conditioning benefits but which is insufficient to interfere with deposition of said dye on said substrate.

21. The method of claim 20, wherein said coloring composition is applied to said keratin-containing substrate for from about 5 to about 10 minutes.

22. The method of claim 20, wherein said keratin-containing substrate is selected from the group consisting of hair, skin, nails, teeth, tissues, wool and fur.

23. A coloring composition for depositing colors to a keratin-containing substrate, said composition comprising:
a) one or more acid dyes;
b) one or more cationic quaternary ammonium compounds, said quaternary ammonium compound being present in an amount which provides conditioning benefits such that the keratin-containing substrate exhibits a combability force of less than about 0.2 joules but which is insufficient to interfere with deposition of said dye on said substrate such that Delta E of said keratin-containing substrate is at least about two units.
